(19) United States
(12) Patent Application Publication
(10) Pub. No.: US 2016/0228805 A1
(43) Pub. Date: Aug. 11, 2016

(54) BREATH SAMPLING FILTER DEVICES AND GAS ANALYZER INCLUDING SAME

(71) Applicant: ORIDION MEDICAL 1987 LTD., Jerusalem (IL)

(72) Inventors: Avner Bar-Lev, Givatayim (IL); Alon Sasson, Rehovot (IL); Michal Ronen, Givat-Brenner (IL); Seamus Maguire, Athlone (IE)

(21) Appl. No.: 14/616,846

(22) Filed: Feb. 9, 2015

(51) Int. Cl. B01D 46/00; G01N 33/497

(52) U.S. Cl. CPC B01D 46/0024; G01N 33/497; B01D 46/0036; B01D 46/0002

(57) ABSTRACT

A filtering device for use in breath sampling including a filter configured to absorb moisture, a first connector configured to allow connection of a user interface to the filtering device, and a second connector configured for connection to the gas analyzer; wherein the filter is essentially flat, such that when the filtering device is connected to a gas analyzer, the filter is in-line with a panel of the gas analyzer; and wherein a circumference of the first and second connectors are smaller than a circumference of the filter.

### TECHNICAL FIELD

The present disclosure generally relates to the field of breath sampling, filter devices and gas analyzers including same.

### BACKGROUND

Accurate monitoring concentrations of a gas, such as for example carbon dioxide (CO₂) in exhaled breath, is vital in assessing the physiologic status of a patient. Breath sampling is generally performed through breath sampling tubes configured to be connected to a patient airway and to a medical device. Liquids are common in patient sampling systems and often accumulate both in the patient airway and in the sampling line tubing.

Filters and filtering devices are typically incorporated into breath sampling systems in order to prevent materials other than gasses to pass through to the gas analyzer thou protecting the gas analyzer from failures. Certain filtering devices are capable of effectively filtering for an extended period of time and may thus remain connected to the gas analyzer while replacing the user interface only.

### SUMMARY

The present disclosure relates to filtering devices and gas analyzers including same configured to enable long-term attachment of the filter device to the gas analyzer while protecting the filtering device from physical damage.

The capability of modern filtering devices to affectively filter for prolonged period of time provides a possibility of long term connection of the filtering device to the gas analyzer while exchanging the user interfaces (e.g. the sampling tube) only. However, a major problem of such long term connection, is the risk of damage caused to the filter due to it being accidently pulled out, hit or damaged, especially in the absence of connection to a patient user interface since the filtering device as currently designed protrudes out from the panel of the gas analyzer.

Advantageously, the filtering devices and/or the gas analyzers disclosed herein are shaped in a way allowing the filtering device to be in-line with the outline of the gas analyzer, so that the filtering device does not stick out. This protects the filtering device from being accidently bumped into or otherwise damaged, even when not in use and thus truly enables long term use of filters while exchanging the user interface only. As filtering devices typically constitute the expensive part of a breath sampling line, such prolonged use of filters may greatly reduce the cost of breath sampling.

In addition, the in-line configuration of the filtering devices with the gas analyzers generates a vicinity between the elements allowing efficient transfer of heat, produced by the gas analyzer, to the filtering device. As a result, the temperature near the filtering device is higher than the surrounding temperature enabling a higher rate of liquid evaporation.

According to some embodiments, there is provided a filtering device for use in breath sampling, the filtering device including a filter configured to deflect and/or absorb moisture, a first connector configured to allow connection of a user interface to said filtering device, and a second connector configured for connection to said gas analyzer.

According to some embodiments, the filter may be essentially flat, such that when connected to a gas analyzer, the filter will be in-line with a panel of the gas analyzer.

According to some embodiments, the first and second connectors may include an inner fluid flow channel configured to allow free flow of breath samples therethrough.

According to some embodiments, the circumference of the first and second connectors are smaller than the circumference of the filter. According to some embodiments, the circumference may be an outer diameter.

According to some embodiments, the filter may include a hydrophobic material at an inner circumference thereof and a hydrophilic material at in outer circumference thereof. According to some embodiments, the ratio of the hydrophobic material and the hydrophilic material may be in a range of 1:1-1:100. According to some embodiments, the ratio of the hydrophobic material and the hydrophilic material may be in a range of 1:10-1:50.

According to some embodiments, the first and second connectors may be positioned on opposite sides of the hydrophobic material, such that breath samples flowing through the inner fluid flow channel of the first connector pass through the hydrophobic material prior to reaching the inner fluid flow channel of the second connector.

According to some embodiments, the hydrophobic material may be sized to cover the entire opening of the inner fluid flow channel of the first and second connectors.

According to some embodiments, essentially flat may include a thickness of less than 1 cm. According to some embodiments, essentially flat may include a thickness of less than 0.5 cm.

According to some embodiments, there is provided a gas analyzer including a filtering device having at least one filter configured to absorb moisture, the filter being essentially flat and in-line with a panel of the gas analyzer and a connector configured to allow connection of a user interface to the filtering device,

According to some embodiments, the circumference of the connector may be smaller than the circumference of the filter.

According to some embodiments, the gas analyzer may further include a socket configured to receive the filtering device such that the filtering device is in-line with the panel of the gas analyzer.

According to some embodiments, the gas analyzer may further include a heat element configured to receive heat produced by the gas analyzer and to direct the heat to the filtering device.

According to some embodiments, the gas analyzer may be a capnograph.

According to some embodiments, there is provided a gas analyzer including a filter-receiving compartment having a connector configured to allow connection of a filtering device, the filter-receiving compartment being sized and shaped to avoid protrusion of the filtering device from the panel of the gas analyzer upon connection of the filtering device to the connector.

According to some embodiments, the filter-receiving compartment may include an indent. According to some embodiments, the filter-receiving compartment may include a socket.

According to some embodiments, the longitudinal axis of the filter-receiving compartment may have a length essentially similar to the length of filtering device along the longitudinal axis thereof.

According to some embodiments, the filtering device may be essentially cylinder shaped.

According to some embodiments, the gas analyzer may further include a heat element configured to receive heat produced by the gas analyzer and to direct the heat to the filter-receiving compartment.

According to some embodiments, the gas analyzer may be a capnograph.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIGS. 1A and 1B schematically illustrate a filtering device, according to some embodiments;

FIG. 2 schematically illustrates a filtering device connected to a gas analyzer according to some embodiments;

FIG. 3 schematically illustrates a gas analyzer having a filter-receiving compartment, according to some embodiments;

FIG. 4 schematically illustrates a gas analyzer having a filter-receiving compartment, according to some embodiments;

FIG. 5 schematically illustrates a gas analyzer having a filter-receiving compartment and a protective door; according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

There is provided, according to some embodiments, a filtering device for use in breath sampling, the filtering device including a filter configured to deflect and/or absorb moisture, a first connector configured to allow connection of a user interface to the filtering device and a second connector configured for connection to the gas analyzer.

As used herein the term “filtering device” may refer to any device including and/or constituting a filter. According to some embodiments, the filtering device may refer to a housing including therein a filter. According to some embodiments, the filtering device may refer to an arrangement including an exposed filter and optionally additional elements such as but not limited to a connector.

As used herein, the term “filter” may refer to any material configured to deflect and/or adsorb liquids, such as but not limited to water, saline, secretions and the like. Each possibility is a separate embodiment. According to some embodiments, the filter may include molecular sieve. As used herein, the term “molecular sieve” may refer to a material with very small holes of precise and uniform size. These holes are small enough to block large molecules, while allowing small molecules to pass. According to some embodiments, the molecular sieves may be utilized to reduce the water content of a gas, such as but not limited to exhaled breath.

According to some embodiments, the filtering device may include more than one filter, such as 2, 3, 4, 5 or more filters. Each possibility is a separate embodiment.

As used herein the term “deflect” may refer to a material configured to repel moisture. According to some embodiments, the material configured to deflect moisture may be a hydrophobic material.

As used herein the term “absorb” may refer to a material configured to attract moisture. According to some embodiments, the material configured to deflect moisture may be a hydrophilic material.

According to some embodiments, the filter and/or the filtering device may be essentially flat. As used herein the term “essentially flat” may refer to filters and/or filtering devices having a thickness (or depth) of less than 1.5 cm, of less than 1 cm, of less than 0.5 cm, of less than 0.25 cm, or of less than 0.1 cm. Each possibility is a separate embodiment. According to some embodiments, when connected to a gas analyzer, the filter and/or the filtering device may be in-line with a panel of the gas analyzer. As used herein, the term “in-line” may refer to a filter and/or filtering device protruding out from the panel of the gas analyzer by less than 1 cm, by less than 0.5 cm or less than 0.25 cm. Each possibility is a separate embodiment.

As used herein, the term “gas analyzer” may refer to any monitor and/or sensor configured to analyze a breathing gas, such as but not limited to a breathing gas. According to some embodiments, the gas analyzer may be a CO₂ sensor. According to some embodiments, the gas analyzer may be a capnograph.

As used herein, the term “user interface” may refer to a sampling tube, an oxygen supply tube, a breath sampling cannula or any other suitable means for transferring breath samples from a subject to a gas analyzer. Each possibility is a separate embodiment.

According to some embodiments, the first and second connectors may include an inner fluid flow channel configured to allow free flow of breath samples there through. According to some embodiments, the inner fluid flow channel may be coextensive between the first and second connectors, separated only by the filter. According to some embodiments, the inner fluid flow channel may allow undisturbed flow of breath from a user interface, such as a sampling tube, through the filter and to the gas analyzer.

According to some embodiments, the circumference of the first and/or second connector may be smaller than the circumference of the filter. According to some embodiments, the filter may be essentially circular. According to some embodiments, the diameter of the filter may be larger than the diameter of the connector.

According to some embodiments, the filter may include a hydrophobic material and a hydrophilic material. According to some embodiments, the inner circumference of the filter may include a hydrophobic material and outer circumference may include a hydrophilic material. According to some embodiments, the ratio of hydrophobic material and hydrophilic material may be in a range of 1:1-1:100, 1:2-1:75, 1:5-1:50, 1:10-1:50, 1:10-1:20 or any other suitable range allowing moisture repelled by the hydrophobic material being adsorbed by the hydrophilic material. Each possibility is a separate embodiment.

According to some embodiments, the first and second connectors may be positioned on opposite sides of the hydrophobic material, such that breath samples flowing through the inner fluid flow channel of the first connector may pass through the hydrophobic material prior to reaching the inner fluid flow channel of the second connector. According to some embodiments, the hydrophobic material may be sized and/or shaped to cover the entire opening of the inner fluid flow channel of the first and second connectors. According to some embodiments, the hydrophobic material may be sized and/or shaped to cover no more than the opening of the inner fluid flow channel of the first and second connectors. According to some embodiments, the hydrophobic material may be sized and/or shaped to cover 70-90% of the opening of the inner fluid flow channel of the first and second connectors. According to some embodiments, the hydrophobic material may be sized and/or shaped to cover 110-120% of the opening of the inner fluid flow channel of the first and second connectors.

It is thus understood that the configuration of the filter may be such that the breath samples directly encounter only the hydrophobic material ensuring that the sample gasses pass through the filter with essentially no disruption to its laminar flow. Liquids, repelled by the hydrophobic material, will on the other hand be absorbed by the hydrophilic material (and/or the molecular sieve) preventing it from clogging the inlet to the gas analyzer.

According to some embodiments, there is provided a gas analyzer, such as but not limited to a capnograph, including a filtering device with a filter configured to deflect and/or absorb moisture and a connector configured to allow connection of a user interface to the filtering device, as essentially described herein.

According to some embodiments, the filter and/or the filtering device may be essentially flat and in-line with a panel of the gas analyzer, as essentially described herein.

According to some embodiments, the gas analyzer may further include a compartment, sized and/or shaped to receive the filtering device such that the filtering device does not protrude out from the panel of the gas analyzer.

According to some embodiments, the gas analyzer may further include a heat element. As used herein, the term “heat element” may refer to any element, wire and/or material configured to receive heat produced by the gas analyzer and to direct the heat to the filtering device or to the vicinity of the filtering device. Each possibility is a separate embodiment. It is understood that the heat element may ensure that the temperature near the filtering device is higher than the surrounding temperature, thereby enabling a higher evaporation rate of liquids.

According to some embodiments, there is provided a gas analyzer, such as bot not limited to a capnograph, including a filter-receiving compartment. According to some embodiments, the filter-receiving compartment may include a connector configured to allow connection of a filtering device. According to some embodiments, the filter-receiving compartment may be sized and shaped to avoid the filtering device from protruding out from the panel of the gas analyzer upon connection of the filtering device to the connector.

According to some embodiments, the filter-receiving compartment may include an indent formed, for example, in the panel of the gas analyzer. According to some embodiments, the filter-receiving compartment may include a socket. As used herein, the term “socket” may refer to a channel or tunnel formed compartment configured to encompass therein a filter and/or a filtering device.

According to some embodiments, the longitudinal axis of the filter-receiving compartment may have a length of a longitudinal axis of the filtering device. According to some embodiments, the longitudinal axis of the filter-receiving compartment may be longer such as 105%, 110% or 120% times the length of the filtering device. Each possibility is a separate embodiment.

According to some embodiments, the filtering device may be essentially cylinder shaped. According to some embodiments, the filtering device may be essentially circular. According to some embodiments, the filtering device may have oval shape, an elliptical shape or any other suitable shape. Each possibility is a separate embodiment.

According to some embodiments, the gas analyzer may further include a heat element configured to receive heat, produced by the gas analyzer, and to direct the heat to the filter-receiving compartment.

According to some additional or alternative embodiments, the gas analyzer may include a protective door(s) configured to protect the filtering device. According to some embodiments, the protective door may be a folding door. According to some embodiments, the protective door may be opened by pressing an activating button. According to some embodiments, the door may include a handle configured to open the door when pulled by a user. According to some embodiments, the protective door may be opened by pressing on the door from the outside. According to some embodiments, the door is configured to be closed automatically, for example by the force of a spring. According to some embodiments, the protective door may include a hinge configured to retain the protective door in an open position once pulled open. According to some embodiments, the protective door may be configured to close off a filter-receiving compartment, such as but not limited to the filter-receiving compartment described herein. According to some embodiments, the protective door may close off the filter-receiving compartment at an end thereof, i.e. in alignment with the panel of the gas analyzer. According to some embodiments, the protective door may be configured to fold around the filtering device, thereby forming a filter receiving compartment. According to some embodiments, the gas analyzer may further include a stopper configured to stop and/or retain the protective door at a predetermined distance from the filtering device.

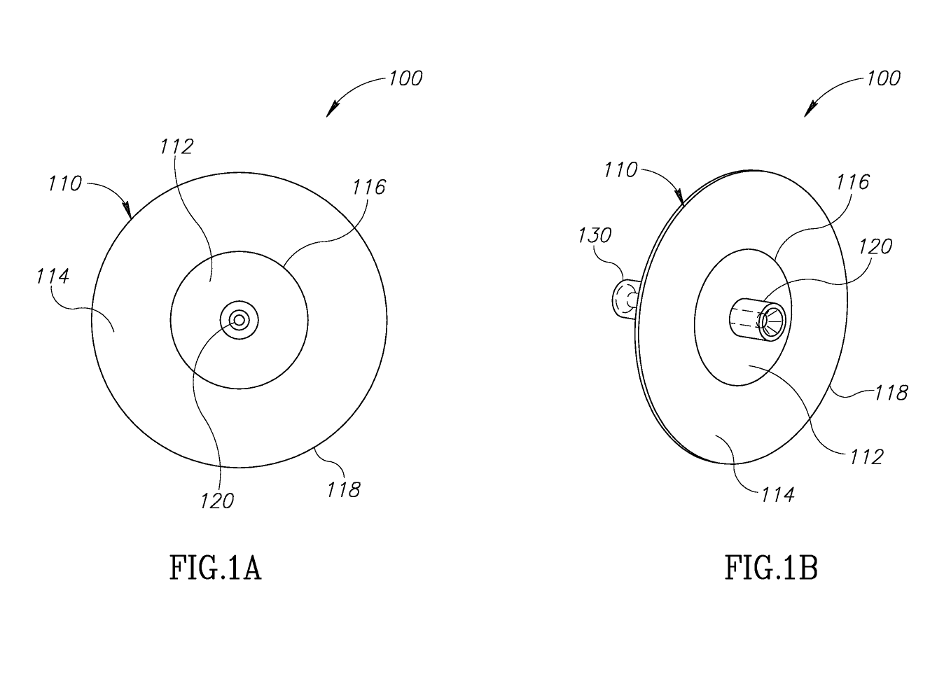

Reference is now made to FIGS. 1A and 1B, which schematically illustrate a front and a perspective view, respectively, of a filtering device 100, according to some embodiments. Filtering device 100 may include a filter 110 configured to deflect and adsorb moisture. Filter 110 is essentially flat and includes a hydrophobic material 112 configured to deflect and/or repel aqueous liquids away, and a hydrophilic material 114 configured to absorb liquids. Within a circumference 116 of hydrophobic material 112 is a first connector 120 configured to allow connection of a user interface (not shown) to filtering device 100. It is thus understood that the circumference of connectors 120 and 130 is smaller than an outer circumference 118 of filter 110. According to some embodiments, the circumference of connectors 120 and 130 is smaller than circumference 116 of hydrophobic material 112. Similarly, on the opposite side of hydrophobic material 112 is a second connector 130 configured for connection to a gas analyzer (not shown). Hydrophobic material 112 is sized and shaped to fit the circumference of connectors 120 and 130. This configuration serves to ensure that breath samples, obtained from the sampling tube, pass through filtering device 100 essentially without disturbing its laminar flow. Liquids present in the breath sample will be repelled by hydrophobic material 112. Consequently, the liquids will be pushed away toward circumference 116 of hydrophobic material 112, thereby facilitating absorption of the liquids by hydrophilic material 114, positioned externally to outer circumference 116 of hydrophobic material 112, and in no direct contact with connectors 120 and 130.

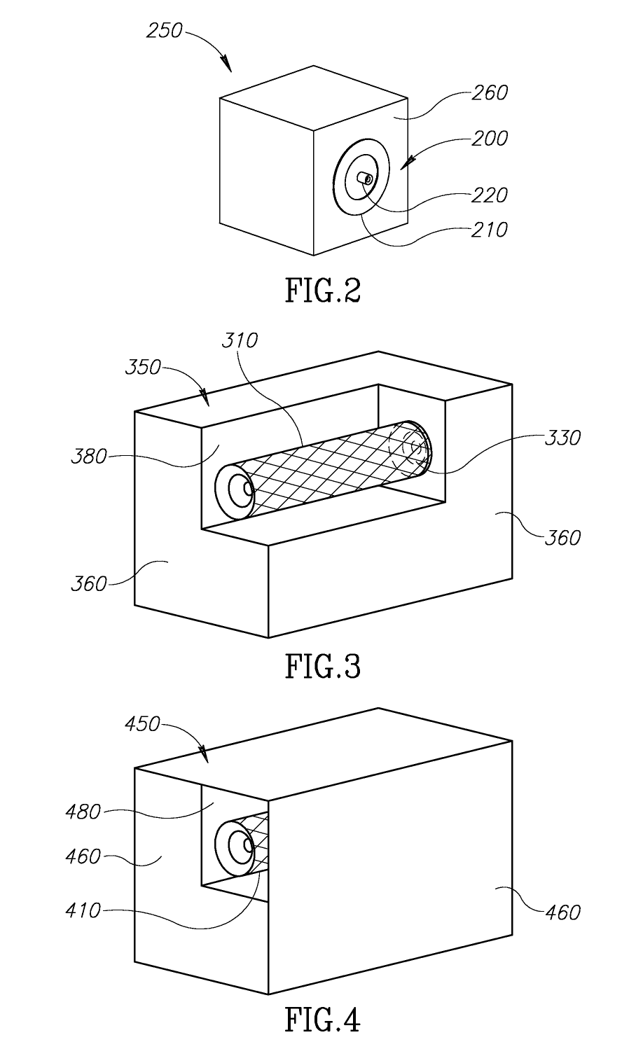

Reference is now made to FIG. 2, which schematically illustrates a filtering device 200 comprising a filter 210, connected to a gas analyzer 250, according to some embodiments. Due to its flat configuration, filter 210 is in-line with panel 260 of gas analyzer 250. Optionally, filtering device 200 may be positioned within a compartment, such as an indentation (not shown). This enables connector 220, configured to allow connection of a user interface (not shown) to filtering device 200, also to be largely in-line with panel 260 of gas analyzer 250. As a result, the entire filtering device 200 is prevented from sticking out from panel 260 of gas analyzer 250, whereby risk of damage during long-term connection to gas analyzer 250 is reduced if not eliminated.

Reference is now made to FIG. 3, which schematically illustrates a gas analyzer 350 (e.g. a capnograph), according to some embodiments. Gas analyzer 350 includes a filter receiving compartment, here an indent 380, including a connector 330 configured to allow connection of a filtering device, here cylindrical filter 310 to gas analyzer 350. This ensures that when cylindrical filter 310 is connected to gas analyzer 350, through connector 330, the entire cylindrical filter 310 will be positioned within indent 380 and will therefore not protrude out from panel 360 of gas analyzer 350. Cylindrical filter 310 will thereby be protected from physical damage.

Reference is now made to FIG. 4, which schematically illustrates a gas analyzer 450 (e.g. a capnograph), according to some embodiments. Gas analyzer 450 includes a filter receiving compartment, here a socket 480, including a connector (not shown) configured to allow connection of a filtering device, here cylindrical filter 410 to gas analyzer 450. This ensures that when cylindrical filter 410 is connected to gas analyzer 450, the entire cylindrical filter 410 is positioned within and encompassed by socket 480. Consequently, cylindrical filter 410 does not protrude out from panel 460 of gas analyzer 450 and will thereby be protected from physical damage.

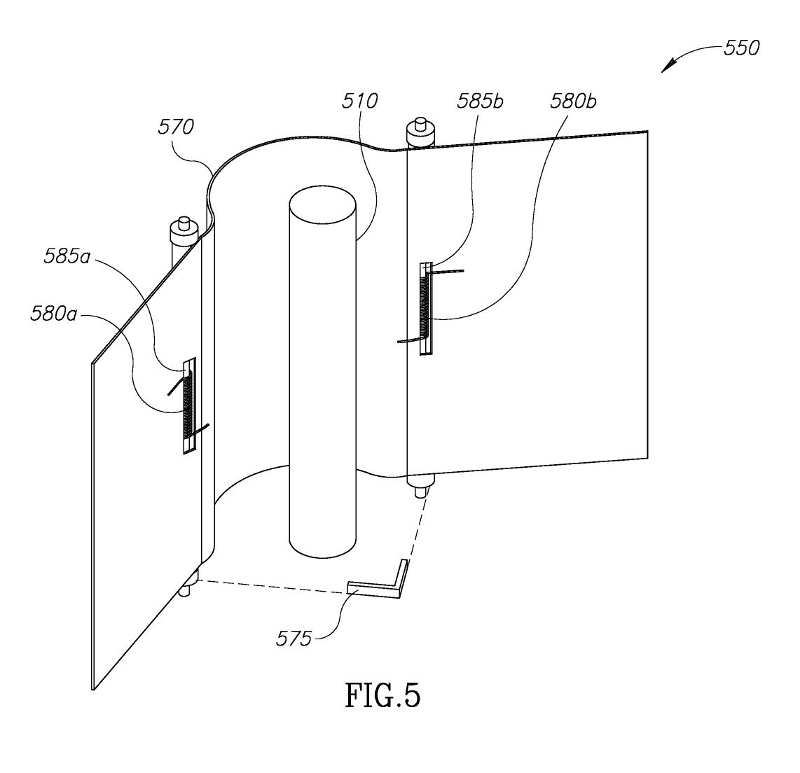

Reference is now made to FIG. 5, which schematically illustrates a filter receiving compartment 550 configured for attachment to (or being an integral part of) a gas analyzer, such as but not limited to a capnograph, according to some embodiments. Filter receiving compartment is configured to receive a filtering device, here cylindrical filter 510. Filter receiving compartment 550 includes a three-sectioned foldable protective door 570 configured to fold around cylindrical filter 510, thereby forming a protective compartment therearound. Filter receiving compartment 550 further includes a triangular stopper 575 configured to stop the folding of protective door 570 and thereby to retain the door at a predetermined distance from cylindrical filter 510. Protective door further include springs 580a and 580b configured automatically to close folding door 570 around cylindrical filter 510, and hinges 585a and 585b configured to retain folding door 570 open, once opened beyond a predetermined angle.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1. A filtering device for use in breath sampling, said filtering device comprising:
a filter configured to deflect and/or absorb moisture, said filter being essentially flat, such that when connected to a gas analyzer, said filter is in-line with a panel of said gas analyzer;
a first connector configured to allow connection of a user interface to said filtering device, and
a second connector configured for connection to said gas analyzer;
wherein said first and second connectors comprise an inner fluid flow channel configured to allow free flow of breath samples therethrough; and
wherein a circumference of said first and second connectors are smaller than a circumference of said filter.

2. The filtering device of claim 1, wherein said circumference is an outer diameter.

3. The filtering device of claim 1, wherein said filter comprises a hydrophobic material at an inner circumference thereof and a hydrophilic material at in outer circumference thereof.

4. The filtering device of claim 3, wherein a ratio of said hydrophobic material and said hydrophilic material is in a range of 1:1-1:100.

5. The filtering device of claim 3, wherein a ratio of said hydrophobic material and said hydrophilic material is in a range of 1:10-1:50.

6. The filtering device of claim 3, wherein said first and second connectors are positioned on opposite sides of said hydrophobic material, such that breath samples flowing through said inner fluid flow channel of said first connector pass through said hydrophobic material prior to reaching said inner fluid flow channel of said second connector.

7. The filtering device of claim 6, wherein said hydrophobic material is sized to cover an entire opening of said inner fluid flow channel of said first and second connectors.

8. The filtering device of claim 1, wherein essentially flat comprises a thickness of less than 1 cm.

9. The filtering device of claim 1, wherein essentially flat comprises a thickness of less than 0.5 cm.

10. A gas analyzer comprising:
a filtering device comprising at least one filter configured to absorb moisture, said filter being essentially flat and in-line with a panel of said gas analyzer; and
a connector configured to allow connection of a user interface to said filtering device, wherein a circumference of said connector is smaller than a circumference of said filter.

11. The gas analyzer of claim 10, further comprising a socket configured to receive said filtering device such that said filtering device is in-line with said panel of said gas analyzer.

12. The gas analyzer of claim 10, further comprising a heat element configured to receive heat produced by said gas analyzer and to direct said heat to said filtering device.

13. The gas analyzer of claim 10, being a capnograph.

14. A gas analyzer comprising a filter-receiving compartment comprising a connector configured to allow connection of a filtering device, said filter-receiving compartment being sized and shaped to avoid protrusion of said filtering device from a panel of said gas analyzer upon connection of said filtering device to said connector.

15. The gas analyzer of claim 14, wherein the filter-receiving compartment comprises an indent.

16. The gas analyzer of claim 14, wherein the filter-receiving compartment comprises a socket.

17. The gas analyzer of claim 14, wherein a longitudinal axis of said filter-receiving compartment has a length of a longitudinal axis of said filtering device.

18. The gas analyzer of claim 14, wherein said filtering device is essentially cylinder shaped.

19. The gas analyzer of claim 14, further comprising a heat element configured to receive heat produced by said gas analyzer and to direct the heat to said filter receiving compartment.

20. The gas analyzer of claim 14, being a capnograph.
